(19) United States
(12) Patent Application Publication
(10) Pub. No.: US 2012/0184022 A1
(43) Pub. Date: Jul. 19, 2012

(54) Convenient System and Method of Composting

(75) Inventor: Leslie H. Power, Clinton Corners, NY (US)

(73) Assignee: Leslie Hamilton Power, Clinton Corners, NY (US)

(21) Appl. No.: 13/007,653

(22) Filed: Jan. 16, 2011

(51) Int. Cl. C12M 1/00; C12S 3/00

(52) U.S. Cl. 435/267; 435/290.1; 435/290.4

(57) ABSTRACT

The present invention describes a system and method for convenient composting. The system is comprised of a door within a building that opens to the outdoors. Additionally, the system comprises a chute proximal to the door, or affixed to a portion of the door wherein the chute can transport compostable materials to a compost bin. The method disclosed in the present invention comprises the steps of composting including using a door to transport material from indoors to outdoors, placing the material on a chute, wherein the material is transported from an indoor location to a compost bin.

### FIELD OF THE INVENTION

This invention relates to a convenient system and method for composting. More specifically, a system and method for transporting compostable material from an indoor location to an outdoor compost area is disclosed.

### BACKGROUND OF THE INVENTION

While the art of composting has existed for many years, there are, at present, few convenient systems and methods available for users to transport compostable materials from an indoor location to an outdoor compost location. Composting is desirable because it allows kitchen waste to be converted into organic material that can be used as fertilizer. Additionally, those who compost reduce the amount of waste from their home that would otherwise end up in a garbage dump. Recently, people have become more conscientious about minimizing the amount of trash they create by utilizing recycling and composting, among other things.

When someone wishes to compost, she typically compiles her compostable materials in a bucket or strainer in their kitchen. When the bucket or strainer becomes full, people typically transport the materials therein to an outdoor location. This system and method is less than desirable because strainers and buckets can sit in the kitchen for some time before they are emptied. Compost materials can attract bugs or vermin and can be odorous if allowed to stay indoors for prolonged periods of time. In addition, when an individual does transport her compost strainer or bucket outdoors, this process s messy because strainers and buckets often drip on the floor or carpet as they are being transported to an outdoor location. During winter months, it is not convenient for people who live in colder climates to carry their compost bucket or strainer to a compost location that is removed from the close environs of their home. Similarly, for those who live in regions with extended periods of rain, transporting compost strainers and buckets outdoors can likewise be burdensome.

Recently, there have been some developments in the area of indoor composting units. These units are typically fitted to be housed in a cabinet in the kitchen thereby allowing users to easily and conveniently compost from indoors without having to carry a bucket or strainer full of compost material to an outdoor composting location. Although these units have increased the convenience associated with composting, they do not provide an easy and convenient system for outdoor composting without ever having to leave the kitchen. There is thus a need for a system and method that allows users to bypass the inconvenience of collecting compost materials in a bucket or strainer for later transportation outdoors.

### SUMMARY OF THE INVENTION

The present invention describes a system and method for convenient composting. The system comprise a door or a window within a building that easily opens to the outdoors. Additionally, the system comprises a chute proximal to the door or window, or affixed to a portion of the door or window wherein the chute can transport compostable materials to a compost bin. The method disclosed in the present invention comprises the steps of composting including using a door or window to transport material from indoors to outdoors, placing the material on a chute, wherein the material is transported from an indoor location to a compost bin.

### BRIEF DESCRIPTION OF THE DRAWINGS

FIG. 1 shows a front elevational view of a door with a chute proximal to a portion thereof.

FIG. 2 is a rear view of a door with a chute proximal to a portion thereof.

FIG. 3 is side view of a chute affixed to a portion of a door or building.

FIG. 4A is a side view of a bin that could be used with the present invention.

FIG. 4B is a side view of an alternate embodiment of a bin that could be used with the present invention.

FIG. 4C is a side view of an alternate embodiment of a bin that could be used with the present invention.

### DETAILED DESCRIPTION OF THE INVENTION

Systems and methods for convenient composting are disclosed. The following description is presented to enable any person skilled in the art to make and use the invention. Descriptions of specific embodiments and applications are provided only as examples and various modifications will be readily apparent to those skilled in the art. The general principles defined herein may be applied to other embodiments and applications without departing from the scope and spirit of the invention. Thus, the present invention os to be accorded the widest scope encompassing numerous alternatives, modifications, and equivalents consistent with the principles and features disclosed herein. For purposes of clarity, details relating to technical material that is known in the technical fields related to the invention have not been described in detail so as to avoid obscuring the present invention. The invention described herein is not limited in its application to the details of construction and the arrangements of the components set forth in the following description or illustrations in the accompanying figures. The use of letters to identify steps of a method or process is simply for identification and is not meant to indicate that the steps should be performed in a particular order.

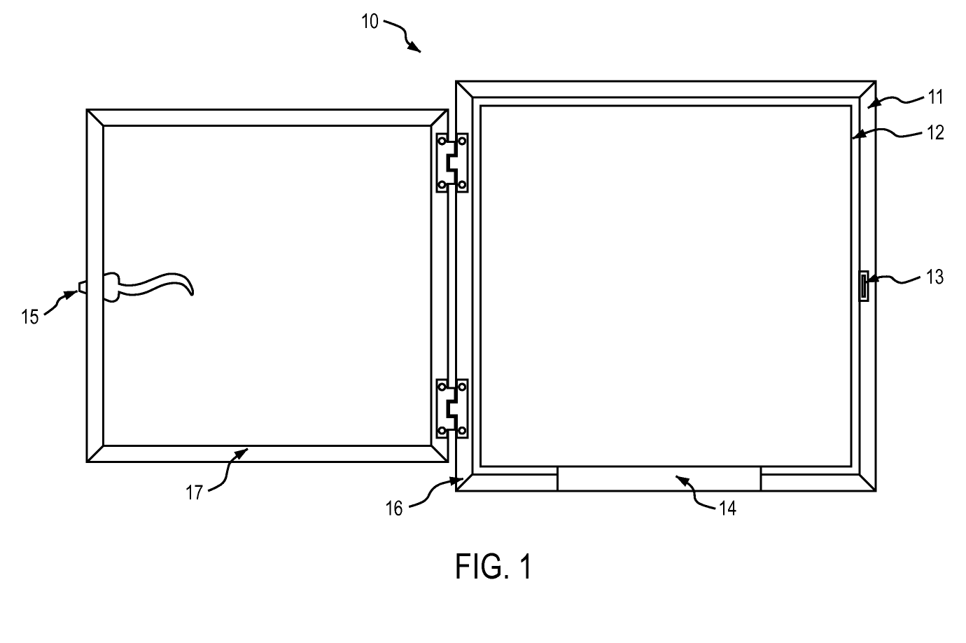

FIG. 1 shows a door 10 in accordance with the present invention. The door may be comprised of a fixed frame 11 affixed to a wall or similar structure, a gasket 12, a latch 13, a top portion of a chute 14, a handle 15, a front panel 16, and a movable frame 17. According to the present invention, the door 10, is located within a building, e.g., a home, restaurant, coffee shop, cafeteria, or similar interior location where people are likely to generate compostable materials. In one embodiment, the door 10 is located in a kitchen near where people prepare food. During the course of preparing dinner, for example, someone may generate vegetable waste, coffee grounds, paper waste, or similar compostable materials. After each of these compostable items is generated, the person utilizing the present invention would open the door 10 and place the compostable matter on the top portion of the chute 14. The compostable matter would then travel down the chute 14 under force of gravity where it would land in a compost bin 41, 42, or 43. In one embodiment, the door 10 can be a window.

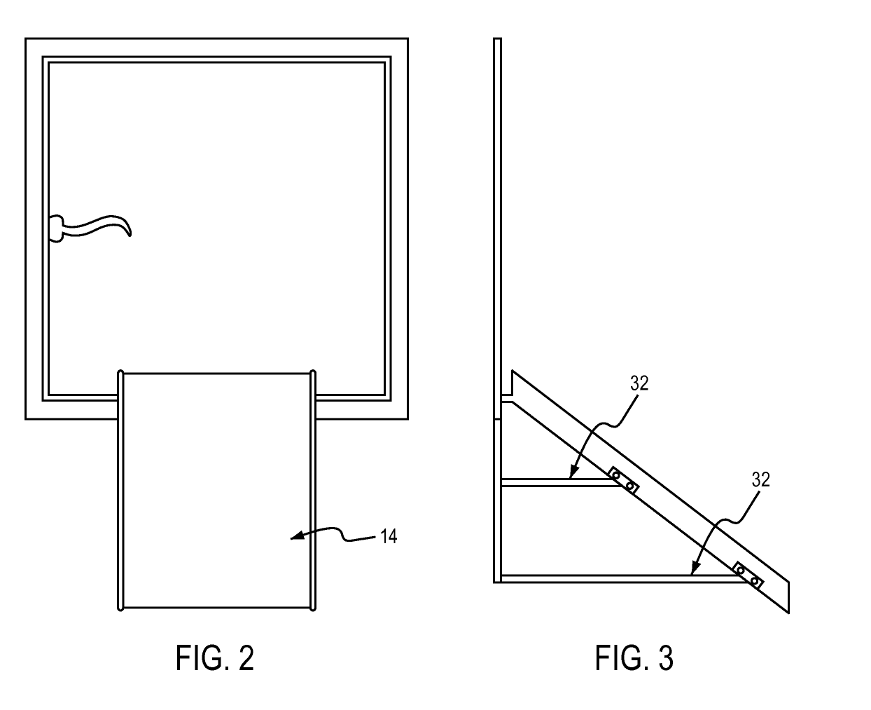

Turning to FIG. 2, in this embodiment, when the compostable matter is placed on the top portion of the chute 14, the compostable matter will travel downward under force of gravity toward the bottom portion of the chute 14. With reference to FIG. 3, it can be seen that top portion of the chute 14 protrudes from the interior space, in this embodiment, from a home. The top portion of the chute 14 is affixed to a portion of the door 10 and is thus accessible when the door 10 is opened. In the embodiment depicted in FIG. 3, the chute 14 is also affixed to the exterior of a building with two brackets 32. The number and location of these brackets can vary in alternate embodiments depending upon the length of the chute 14, the weight of the compostable materials to be transported via the chute 14, and other well known metrics readily apparent to those skilled in the art.

In one embodiment the chute 14 could be made of stainless steel. Additional embodiments of the chute 14 could be comprised of aluminum, Nuovinox, carbon steel rebar clad with stainless steel, titanium, plastic, acrylic, or similar materials that will withstand exposure to the elements without rusting or deteriorating. Similarly, the door 10 in one embodiment could have a transparent pane thereby creating a window. The transparent pane could be constructed of various types and grades of glass as is well known in the art.

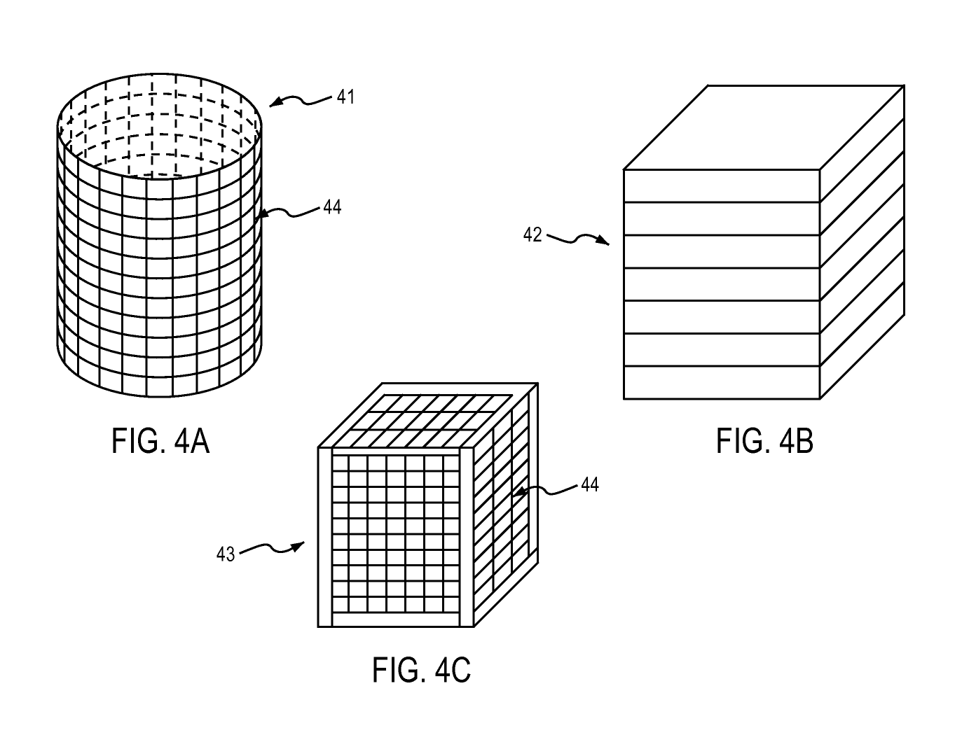

The compost bin 41, 42, or 43 could be made of any suitable material and in many shapes and sizes, as is well known in the art. FIG. 4 shows three exemplary embodiments of compost bins. The compost bin 41 is generally circular in shape and is made of wire 44. The wire 44 could be chicken wire, mesh, wire fencing or similar material as is well known in the art. In terms of choosing the type of wire 44 to use, considerations that are well known in the art, such as tensile strength and the area of the holes would likely be considered. The tensile strength would likely be sufficient enough to stand on its own or with the aid of supporting brackets. Similarly, the area of the holes would be large enough to allow air to flow through, because this expedites the decomposition process, while being small enough to contain the compostable material housed therein.

FIG. 4A shows an elliptical compost bin 41 made of wire 44. FIG. 4B shows a compost bin 42 that is cubical. In this embodiment, the compost bin 42 is made of wood. Those skilled in the art will recognize that the compost bin 42 could be made of metal, plastic, or similar material. FIG. 4C is a cubical compost bin 43 that has wire 44 on four sides. The addition of the wire 44 in this embodiment expedites the decomposition process. In terms of dimensions of the compost bin 41, 42 or 43, the dimensions can vary. Some considerations that may factor into determining the dimensions of the compost bin 41, 42, or 43 are the volume of compostable material likely to accumulate, the amount of time required for the material to decompose, whether an individual would like to access the contents of the compost bin 41, 42, or 43. In some instances, individuals may want to stir the contents of the compost bin 41, 42, or 43 in order to expedite decomposition. Similarly, an individual may want to access the interior of the compost bin 41, 42, or 43 in order to extricate useable fertilizer. In some embodiments, the user may desire a compost bin that is small enough to transport. The compost bins depicted in FIGS. 4A-4C could be scaled to achieve this goal or could include a door that opens thereby .

The present invention lends itself to a method of using the above-described system for convenient composting. More particularly, a method for outdoor composting of materials that does not require the composter to leave the house could be realized by the present invention by opening a door, placing compostable material on a chute wherein the chute transports the compostable material to an externally located compost bin. The transportation of the compostable material could be accomplished under force of gravity or through mechanically assisted means, such as a conveyor belt. In addition, lubricant could be added to the chute to reduce the coefficient of friction thereby enhancing the force of gravity downward into the compost bin.

I claim:

1. A system for composting indoor organic waste comprising:
a door;
a chute wherein one end of the chute is proximal to a portion of the door; and
a compost bin configured to receive compost material from the chute.

2. The system of claim 1, wherein the door has a pane.

3. The system of claim 1, wherein the door is a window.

4. The system of claim 1, wherein the chute is affixed to a door frame.

5. The system of claim 1 wherein the chute is affixed to an exterior portion of a building.

6. The system of claim 1, wherein the chute is a conveyor belt.

7. The system of claim 1 wherein the compost bin further comprises wire.

8. The system of claim 1 wherein the compost bin is portable.

9. A method of composting indoor organic waste comprising the steps of:
a. opening a door; and
b. placing compostable material on a chute, wherein the chute transports the compostable material to an externally located compost bin.
